(19) United States
(12) Patent Application Publication
(10) Pub. No.: US 2007/0032480 A1
(43) Pub. Date: Feb. 8, 2007

(54) Treatment of ischemia-induced arrhythmias

(75) Inventors: Brian O'Rourke, Sparks, MD (US); Miguel A. Aon, Baltimore, MD (US); Fadi G. Akar, Baltimore, MD (US); Eduardo Marban, Lutherville, MD (US); Gordon Tomaselli, Timonium, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/454,147

(22) Filed: Jun. 14, 2006

Related U.S. Application Data
(60) Provisional application No. 60/690,285, filed on Jun. 14, 2005.

(51) Int. Cl. A61K 31/5513; A61K 31/4745; A61K 31/5377; A61K 31/138; C12Q 1/00

(52) U.S. Cl. 514/221; 514/651; 514/305; 514/232.8; 514/533; 435/4

(57) ABSTRACT

New methods and compositions are provided for preventing development of arrhythmias associated with ischemia and repurfusion. Preferred methods of the invention include treatment to inhibit the mitcohondrial inner membrane anion channel.

The present application claims the benefit of U.S. provisional application No. 60/690,285 filed Jun. 14, 2005, which is incorporated herein by reference in its entirety.

### STATEMENT OF U.S. GOVERNMENT SUPPORT

Funding for the present invention was provided in part by the Government of the United States by virtue of a National Institutes of Health grant. Accordingly, the Government of the United States may have certain rights in this invention.

### FIELD OF THE INVENTION

New methods and compositions are provided for preventing development of arrhythmias associated with ischemia and repurfusion. Preferred methods of the invention include treatment to inhibit the mitochondrial inner membrane anion channel.

### BACKGROUND OF THE INVENTION

Sudden Cardiac Death is a leading cause of death in the United States. The majority of the estimated 250,000-400,000 sudden deaths per year occur in patients with no clinically recognized heart disease. Of all of sudden cardiac deaths, an estimated 75% occur in the setting of coronary artery disease. The current strategy to prevent death due to cardiac arrest is electrical defibrillation, which is applied only after a potentially fatal arrhythmia has begun. Moreover, electrical defibrillation also can be ineffective in restoring normal cardiac electrical function.

Still, it remains that no treatment is available for preventing the development of arrhythmias associated with ischemia and reperfusion.

Reperfusion following pathological and/or clinical occurrence of myocardial ischemia can lead to perturbations in cardiac rhythm, including lethal ventricular arrhythmias, and post-ischemic contractile dysfunction. The clinical occurrence and possible lethal consequences of reperfusion arrhythmias and depressed contractile function have elicited considerable interest in determining the mechanisms underlying the events, and in developing therapeutic approaches for their control.

Ischemia and reperfusion of the heart can lead to many biochemical, ion homeostasis, and ion channel alterations that may contribute to post-ischemic contractile and electrical dysfunction, and serve as a substrate for fatal arrhythmias (1). Several mechanistic hypotheses, such as extracellular K⁺ accumulation (2), depression of gap-junctional conductance (3) and dispersion of action potential (AP) repolarization (4) have emerged as dominant paradigms to explain the genesis of arrhythmias upon reperfusion, but the sequence of cellular events underlying post-ischemic electrical instability has not been elucidated. Sarcolemmal ATP-sensitive K⁺ (K_{ATP}) channels are thought to mediate AP shortening during ischemia and may contribute to post-ischemic electrical heterogeneity, but how these channels are activated and whether they contribute to, or retard functional electrical recovery upon reperfusion are unresolved issues.

Because K_{ATP} channels are metabolic sensors, their role in post-ischemic electrical dysfunction is likely to depend on mitochondrial bioenergetics. Various forms of metabolic stress lead to depolarization of the mitochondrial inner membrane potential (5-8), and post-ischemic conditions, including cellular Ca²⁺ overload and an increase in the production of reactive oxygen species (ROS), favor the degradation of mitochondrial integrity (9-11), leading to necrotic or apoptotic cell death (12). The activation of energy-dissipating channels on the inner membrane, including the mitochondrial permeability transition pore (PTP), has been proposed to mediate cell death during reperfusion (13-15); however, other studies have shown that the PTP inhibitor cyclosporin A (CsA) delays, but does not prevent the loss of, mitochondrial inner membrane potential (ΔΨₘ) in the post-ischemic heart (16).

Metabolic stress, in the form of substrate deprivation (7, 17) or localized ROS generation (8), can trigger cell-wide oscillations or collapse of ΔΨₘ in isolated cardiomyocytes. Further, coordinated cell-wide oscillations in the mitochondrial energy state of heart cells can be induced by a highly localized perturbation of a few elements of the mitochondrial network. This provides evidence in support of a direct connection between loss of mitochondrial function, the K_{ATP} channel, and alterations in the cellular action potential (AP) (see also U.S. Pat. No. 6,521,617), and thus provides a mechanistic advance towards understanding the basis of ischemia-related arrhythmias.

Therefore, it would be desirable to have new methods to prevent and treat Sudden Cardiac Death and the development of arrhythmias associated with ischemia and reperfusion.

### SUMMARY OF THE INVENTION

In one aspect, therapies are provided that can respond to events associated with ischemia that may cause mitochondrial and energeric dysfunction and thereby provide effective stabilization of cardiac electrical activity. In particular, therapies of the invention can prevent or inhibit initiation and/or continuance of arrhythmias, including ischemic-related arrhythmias.

Methods of the invention include treatment of mammalian cells, particularly mammalian cardiac cells such as primate cardiac cells and especially human cardiac cells with one or more compounds that can target (modulate) the mitochondrial inner membrane channel (IMAC), particularly compounds that can effectively inhibit the mitochondrial inner membrane channel, as may be suitably assessed by in vitro assay as disclosed herein.

Preferred compounds for use in therapies of the invention include those that are referred to herein as “IMAC inhibitor compounds” or other similar terms and can be identified e.g. by in vitro assays disclosed herein, including a mitochondrial oxidation assay as disclosed and defined below. Specifically preferred IMAC inhibitor compounds for use in therapies of the invention include benzodiazepine receptor ligands 4′-chlorodiazepam (4′-Cl-DZP) and the isoquinoline carboxamide PK11195, in addition to the IMAC inhibitors reported by Beavis (J. Biological Chemistry 262:15085-15093, 1987) including: amiodarone, amitryptyline, imipramine, dibucaine, propranolol, quinine, clonazepam, bupivacaine, etidocaine, pindolol, and timolol.

IMAC inhibitor compounds, including benzodiazepine receptor ligands, are useful according to the methods of the invention. Benzodiazepines are widely used clinically for their central nervous system effects, which are primarily mediated through their interaction with central BzRs. A second receptor for benzodiazepines is found in peripheral tissues, and is abundant in mitochondrial membranes of most cells (22). The pharmacology of the mitochondrial benzodiazepine receptor has been shown to be different from the central receptor.

Preferred methods of the invention can include identifying a subject that is suffering from or susceptible to an ischemic-related arrhythmia and administering to that subject one or more IMAC inhibitor compounds.

A subject suffering from or susceptible to an ischemic-related arrhythmia may suffer from sudden cardiac death, characterized by unexpected and instantaneous death of a non-traumatic nature, such as tachyarrhythmia. Altematively, the subject may suffer complications of a sustained arrhythmic episode, for example sustained ventricular tachycardia.

Preferred methods of the invention may further include identifying a compound as an IMAC inhibitor compound and administering the identified compound to a patient in need of such treatment, e.g., a patient suffering from or susceptible to ischemic arrhythmia.

In preferred aspects, one or more IMAC inhibitor compounds are administered to ischemic tissue, particularly ischemic cardiac tissue.

Preferably, the compounds of this invention are administered prior to, during or shortly after, cardiac surgery or non-cardiac surgery.

In a particularly preferred aspect, one or more IMAC inhibitor compounds are administered to prevent perioperative myocardial ischemic injury.

Ischemic damage also may occur e.g. during organ transplantation. Thus, one or more IMAC inhibitor compounds may be administered prior to, during and/or following an organ transplantation procedure.

Preferably, the compounds of this invention are administered prophylactically and may be administered locally, e.g. to cardiac tissue.

In a further preferred aspect, methods are provided to reduce myocardial tissue damage (e.g., substantially preventing tissue damage, inducing tissue protection) during surgery (e.g., coronary artery bypass grafting (CABG) surgeries, vascular surgeries, percutaneous transluminal coronary angioplasty (PTCA) or any percutaneous transluminal coronary intervention (PTCI), organ transplantation, or other non-cardiac surgeries) comprising administering to a mammal, particularly a human, one or more IMAC inhibitor compounds.

In a further preferred aspect, methods are provided to reduce myocardial tissue damage (e.g., substantially preventing tissue damage, inducing tissue protection) in patients presenting with ongoing cardiac (acute coronary syndromes, e.g. myocardial infarction or unstable angina) or cerebral ischemic events (e.g. stroke) comprising administering to a mammal, particularly a human, one or more IMAC inhibitor compounds.

In a yet further preferred aspect, methods are provided to reduce myocardial tissue damage (e.g., substantially preventing tissue damage, inducing tissue protection) in a patient with diagnosed coronary heart disease (e.g. previous myocardial infarction or unstable angina) or patients who are at high risk for myocardial infarction (age greater than 60 or 65 and two or more risk factors for coronary heart disease) comprising administering to a mammal, particularly a human, one or more IMAC inhibitor compounds.

In a still further aspect, methods are provided treating cardiovascular diseases such as arteriosclerosis, hypertension, angina pectoris or cardiac hypertrophy comprising administering to a mammal, particularly a human, one or more IMAC inhibitor compounds.

As indicated, the methods of the invention include both acute and chronic therapies.

For example, one or more IMAC inhibitor compounds can be immediately administered to a patient (e.g. i.p. or i.v.) that has suffered or is suffering from a cardiac arrhythmia. Such immediate administration preferably would entail administration of one or more IMAC inhibitor compounds within about 0.1, 0.25, 0.5, 1 or 2 hours, after a subject has suffered from cardiac arrhythmia.

Long-term administration of one or more IMAC inhibitor compounds also will be beneficial, e.g., to subjects that are at greater risk for suffering from a cardiac arrhythmia, such as a patient at an age greater than 60 or 65 and having two or more risk factors for coronary heart disease. For example, one or more IMAC inhibitor compounds can be administered regularly to such patient for at least 2, 4, 6, 8, 12, 16, 18, 20 or 24 weeks, or longer such 6 months, 1 years, 2 years or more. An oral dosage formulation may be preferred for such long-term administration.

This invention is also directed to pharmaceutical compositions which comprise a therapeutically effective amount of one or more IMAC inhibitor compounds.

Other aspects of the invention are disclosed infra.

### BRIEF DESCRIPTION OF THE DRAWINGS

FIG. 1 (A-D). Blockade of mitochondrial oscillations and stabilization of the cellular AP by 4′-Cl-DZP. Freshly isolated cardiomyocytes were loaded with TMRM (100 nM) at 37° C. and patched under voltage-clamp conditions on the stage of the microscope as described in materials and methods. Panel (A) shows the reversible effect of acutely added 4′-Cl-DZP (32 μM) on mitochondrial ΔΨₘ oscillations. Panel (B) depicts mitochondrial oscillations in ΔΨₘ and the sarcolemmal APD were triggered after a highly localized laser flash (3 min before the train of oscillating APDs shown in this panel). Action potentials evoked by brief current injections were recorded during the oscillations. It has previously been shown that during a synchronized cell-wide depolarization-repolarization cycle, the action potential shortens in synchrony with fast mitochondrial depolarization (4). During the APD oscillations: (i) the cell becomes inexcitable in the fully depolarized state (remaining upward spikes are from the stimulus only) (panel C) and (ii) after addition of 64 μM 4′-Cl-DZP the APD oscillations are eliminated with coincident restoration of a stable AP (panel D) and interruption of mitochondrial oscillations with ΔΨₘ recovery.

FIGS. 2 (A and B). Panel (A) depicts ischemia-induced APD shortening in control hearts and hearts treated with varying concentrations of 4′-Cl-DZP. Panel (B) shows representative action potentials from a control and a 100 μM 4′-Cl-DZP treated heart recorded at various intervals during the ischemia protocol. At baseline, APD of control and 100 μM 4′Cl-DZP treated hearts were comparable.

FIGS. 3 (A and B). Panel (A) shows ischemia-induced APD shortening in control hearts and hearts treated with varying concentrations of 4′Cl-DZP (100 μM, 64 μM, 40 μM and control). Panel (B) shows representative action potentials from a control and a 100 μM 4′-Cl-DZP treated heart recorded at various intervals during the ischemia protocol.

FIGS. 4 (A and B). FIG. 4 depicts ischemia-induced APD shortening and representative APs recorded from hearts pre-treated with FGIN-1-27. Panel (A) shows progressive reduction in the AP duration (APD) during the first 10 min of ischemia in control and FGIN-1-27 treated hearts. FGIN-1-27 treated hearts exhibited a more enhanced reduction of action potential amplitude compared to control treated hearts. (B) Comparison of the normalized action potential amplitude and dF/dt after 10 min of ischemia compared to pre-ischemic baseline perfusion in untreated control hearts and hearts treated with FGIN-1-27.

FIG. 5 (A-C). Panel (A) shows incidence of reperfusion-related arrhythmias in all groups. Panel (B) shows representative AP traces during arrhythmias in control and FGIN-1-27 treated hearts. Panel (C) depicts the average heart rate of reperfusion-related arrhythmias in control and FGIN-1-27 treated preparations.

FIGS. 6 (A and B) depicts representative AP traces recorded in a control heart (panel A) and a heart pre-treated with 4′-Cl-DZP (panel B) at various time points during ischemia and reperfusion.

FIGS. 7 (A and B). Panel (A) shows representative APs during recovery upon reperfusion in control, 4′-Cl-DZP, CsA, and FGIN-1-27 treated hearts. Panel (B) shows recovery of APD after 5 min of reperfusion as a percentage of baseline APD in hearts treated with 64 μM 4′Cl-DZP and various concentrations of CsA, indicating that the optimal concentration was 0.2 μM.

### DETAILED DESCRIPTION OF THE INVENTION

As discussed above, preferred therapies of the invention can prevent or inhibit initiation and/or continuance of arrhythmias, including ischemic-related arrhythmias. Particular preferred therapies of the invention include the recovery of mitochondrial inner membrane potential, a key determinant in post-ischemic recovery of the heart.

Preferred methods of the invention include administration of one or more mitochondrial inner membrane channel (IMAC) inhibitor compounds to cells or a subject.

IMAC inhibitor compounds may be suitably identified by a mitochondrial fluorescence assay, detected as disclosed in U.S. Pat. No. 6,183,948 to Marban et al. such as by oxidation of nicotinamide adenine dinucleotide (NADH) or flavin adenine dinucleotide (FAD) moieties, or detected as a loss of mitochondrial inner membrane potential, or an increase in reactive oxygen species production using standard fluorescent indicators, as described in Aon et al, 2003 (Journal of Biological Chemistry 278; 44735-44734, 2003) by means of e.g. fluorescence microscopy, photometry and photographic film, which can include the following assay of steps a) through d) as defined and referred to herein as a “standard mitochondrial fluorescence assay”:
a) provide a population of eukaryotic cells;
b) contact cells with one or more compounds that are candidates as inhibitors of IMAC;
c) contact a second portion of the cells with a known inhibitor of IMAC (such as 4′Cl-DZP); and
d) subject first and second portions of cells to defined oxidative stress (which could include, but is not limited to, local laser-induced oxidation, general light-induced stress, treatment with free radical donors, depletion of the cellular free radical scavenger capacity or inhibition of free radical scavenging enzymes) and measure the ability of the compounds set forth in steps b) and c) to prevent mitochondrial fluorescence changes (i.e., loss of membrane potential, oxidation of the mitochondrial redox indicators, or increases in reactive oxygen species) induced by said oxidative stress, as compared with untreated controls.

Preferably, the above assay will identify a candidate IMAC inhibitor compound that prevents mitochondrial depolarization, NADH or FAD oxidation, and/or reactive oxygen species production by a detectable amount (e.g. as determined by fluorescent microscopy) relative to control cells subjected to oxidative stress in a mitochondrial fluorescence assay as set forth in steps a) through d). A control can be run as the same assay but where the candidate compound has not been exposed to test cells. In particular, preferably an IMAC inhibitor compound will be identified that prevents mitochondrial depolarization, NADH or FAD oxidation, and/or reactive oxygen species production by at least about 10%, 20%, 30%, 40%, 50%, 60%, 70%, 80%, 90% or 100% in a mitochondrial fluorescence assay as set forth in steps a) through d) immediately above relative to a control (i.e. the same assay where the candidate compound has not been exposed to test cells).

IMAC inhibitor compounds may also be suitably identified by a second mitochondrial fluorescence assay, detected as disclosed in U.S. Pat. No. 6,183,948 to Marban et al. such as by oxidation of nicotinamide adenine dinucleotide (NADH) or flavin adenine dinucleotide (FAD) moieties, or detected as a loss of mitochondrial inner membrane potential, or an increase in reactive oxygen species production using standard fluorescent indicators, as described in Aon et al, 2003 (Journal of Biological Chemistry 278; 44735-44734, 2003) by means of e.g. fluorescence microscopy, photometry and photographic film, which can include the following assay of steps a) through d) as defined and referred to herein as a “secondary mitochondrial fluorescence assay”: provide a population of eukaryotic cells; expose untreated control cells to defined oxidative stress (which could include, but is not limited to, local laser-induced oxidation, general light-induced stress, treatment with free radical donors, depletion of the cellular free radical scavenger capacity or inhibition of free radical scavenging enzymes) to induce mitochondrial depolarization, NADH or FAD oxidation, and/or reactive oxygen species production by a detectable amount (e.g. as determined by fluorescent microscopy); and contact cells with one or more compounds that are candidates as inhibitors of IMAC and measure the ability of the compounds to reverse mitochondrial fluorescence changes (i.e., loss of membrane potential, oxidation of the mitochondrial redox indicators, or increases in reactive oxygen species) induced by oxidative stress as set forth in step b) above.

Preferably, the above secondary mitochondrial fluorescence assay will identify a candidate IMAC inhibitor compound that reverses mitochondrial depolarization, NADH or FAD oxidation, and/or reactive oxygen species production by a detectable amount (e.g. as determined by fluorescent microscopy) relative to control cells subjected to oxidative stress in a secondary mitochondrial fluorescence assay as set forth in steps a) through c). A control can be run as the same assay but where the candidate compound has not been exposed to test cells. In particular, preferably an IMAC inhibitor compound will be identified that reverses mitochondrial depolarization, NADH or FAD oxidation, and/or reactive oxygen species production by at least about 10%, 20%, 30%, 40%, 50%, 60%, 70%, 80%, 90% or 100% in a secondary mitochondrial fluorescence assay as set forth in steps a) through c) immediately above relative to a control (i.e. the same assay where the candidate compound has not been exposed to test cells).

The term “candidate compound” or “candidate IMAC inhibitor compound” or other similar term as used herein refers to any chemical compound that can be added to a eukaryotic cell, and may comprise a compound that exists naturally within the cell or is exogenous to the cell. The compounds include native compounds or synthetic compounds, and derivatives thereof. As discussed above, particularly preferred IMAC inhibitor compounds include the peripheral benzodiazepine ligands 4′-chlorodiazepam and PK11195, as well as other IMAC inhibitors including amiodarone, amitryptyline, imipramine, dibucaine, propranolol, quinine, clonazepam, bupivacaine, etidocaine, pindolol, and timolol.

As discussed above, it can be preferred that one or more IMAC inhibitor compounds are administered to prevent perioperative myocardial ischemic injury.

In one aspect of such therapies myocardial tissue damage is reduced during surgery.

In another aspect of such therapies myocardial tissue damage is reduced in patients presenting with ongoing cardiac or cerebral ischemic events.

In another aspect of such therapies, one or more IMAC inhibitor compounds can be administered to cells or a subject to treat chronic ischemia. In addition to acute modulation, ion channels undergo changes on a longer time scale (1). Chronic administration of IMAC inhibitor(s) to decrease ischemic response, for example AP shortening and membrane inexcitability, may be advantageous in subjects with chronic ischemia.

In yet another aspect of such therapies myocardial tissue damage is reduced by chronic administration of one or more IMAC inhibitor compounds to a patient with diagnosed coronary heart disease.

The term “mitochondrial criticality” describes the state of the mitochondrial network just prior to cell-wide depolarization of ΔΨₘ, when the system becomes very sensitive to even small perturbations in conditions (20). In this state, the mitochondrial network of the cardiac cell is pushed to a critical state by metabolic or oxidative stress. The mechanism involves the regenerative activation of IMAC by mitochondrial ROS-induced ROS release. Mitochondrial depolarization could be suppressed either by inhibiting the production of ROS by the electron transport chain, enhancing the ROS scavenging capacity of the cell, or blocking IMAC. Oscillations in the mitochondrial inner membrane potential were closely linked to the activation of the sarcolemmal KATP current, which is a consequence of accelerated ATP hydrolysis by the uncoupled mitochondria, and results in shortening or elimination of the cellular AP. Accordingly, these mechanisms may account for the alteration in the electrophysiology of hearts subjected to ischemia-reperfusion.

In another aspect of such therapies, one or more IMAC inhibitor compounds can be administered to cardiac cells to modulate membrane potential. If ischemia-reperfusion related electrophysiological alteration and arrythmia in intact hearts are in part a consequence of the failure of the cellular mitochondrial network to maintain the mitochondrial inner membrane potential, then a preferred method of the invention uses IMAC inhibitor compounds to modulate membrane potential.

Ischemia is characterized by progressive shortening of action potential duration (APD), and progressive reduction of action potential amplitude (APA) and AP upstroke. According to the methods of the instant invention, IMAC inhibitor compounds, more specifically benzodiazepine receptor ligands, can be administered to provide a reduction in membrane excitability and/or regions of slowed conduction. More specifically, in an aspect of therapy, IMAC inhibitors can be used to treat ischemia-induced APD shortening and provide a reduction in ischemia induced APA and upstroke velocity.

In one particular example, the benzodiazepine receptor ligands 4′-Cl-DZP and FGIN-1-27 are used during ischemia. While 4′-Cl-DZP blunted APD shortening during ischemia, it did not abolish APs. FGIN-1-27 treatment accelerated APD shortening, provided a reduction in AP amplitude and the time for the onset of AP, and slowed conduction velocity (CV) compared to control hearts

In another particular example, the benzodiazepine receptor ligands 4′-Cl-DZP and FGIN-1-27 can be used during reperfusion. 4′CL-DZP prevented reperfusion-related ventricular fibrillation. FGIN-1-27 treatment resulted in long period of electrical silence followed by ventricular tachycardia (VT) of long duration.

In another example, KATP channel blockade is used to blunt the effects of FGIN-1-27, APD shortening and reduced AP amplitude and upstroke velocity that occurs during the beginning of the ischemic response. In a related example the KATP channel blockade is shown to be effective in preserving electrical excitability only within a defined time, for example not beyond 20 minutes of ischemia. Thus, the instant invention further characterizes different responses in membrane excitability in both ischemia and perfusion to different benzodiazeprene receptor ligands.

Typical subjects for treatment include mammals particularly male and female humans that are suffering from or susceptible to ischemia-related arrhythmia.

Preferably, therapies of the invention can provide for myocardial protection before, during, or after coronary artery bypass grafting (CABG) surgeries, vascular surgeries, percutaneous transluminal coronary angioplasty (PTCA), organ transplantation, or non-cardiac surgeries.

Preferred therapies of the invention provide for myocardial protection in patients presenting with ongoing cardiac (acute coronary syndromes, e.g. myocardial infarction or unstable angina) or cerebral ischemic events (e.g. stroke).

The amount and timing of compounds administered will, of course, be dependent on the subject being treated, on the severity of the affliction, on the manner of administration and on the judgment of the prescribing physician. Thus, because of patient-to-patient variability, the dosages given below are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases (e.g., cardiovascular disease).

Thus, for example, in one mode of administration the compounds of this invention may be administered just prior to surgery (e.g., within twenty-four hours before surgery for example cardiac surgery) during or subsequent to surgery (e.g., within twenty-four hours after surgery) where there is risk of myocardial ischemia. The compounds of this invention may also be administered in a chronic daily mode.

Compounds for use in the methods of the invention can be administered in a continuous dose prior to, during or after ischemia. Compounds for use in the methods of the invention can also be administered in a bolus dose, prior to the onset of reperfusion. The bolus dose can be administered, for example at 0, 5, 10, 15 minutes prior to reperfusion.

Compounds for use in the methods of the invention can be administered intranasally, orally (ingestion or sublingual) or by injection, e.g., intramuscular, intraperitoneal, subcutaneous or intravenous injection, or by transdermal, intraocular or enteral means. The optimal dose can be determined by conventional means. Compounds for use in the methods of the invention are suitably administered to a subject in the protonated and water-soluble form, e.g., as a pharmaceutically acceptable salt of an organic or inorganic acid, e.g., hydrochloride, sulfate, hemi-sulfate, phosphate, nitrate, acetate, oxalate, citrate, maleate, mesylate, etc.

IMAC inhibitor compounds can be employed, either alone or in combination with one or more other therapeutic agents, suitably as a pharmaceutical composition in mixture with conventional excipient.

Additional therapeutic agents to administer in conjunction with one or more IMAC inhibitor compounds include e.g. other cardiovascular agents known to those skilled in the art for example β-blockers (e.g., acebutolol, atenolol, bopindolol, labetolol, mepindolol, nadolol, oxprenol, pindolol, propranolol, sotalol), calcium channel blockers (e.g., amlodipine, nifedipine, nisoldipine, nitrendipine, verapamil), potassium channel openers, adenosine, adenosine agoinists, ACE inhibitors (e.g., captopril, enalapril), angiotensin receptor blockers (e.g. losartan, valsartan, ibesartan, candesartan, telmisartan, olmesartan, eprosartan), nitrates (e.g., isosorbide dinitrate, isosorbide 5-mononitrate, glyceryl trinitrate), diuretics (e.g., hydrochlorothiazide, chlorthalidone, furosemide, indapamide, piretanide, xipamide), glycosides (e.g., digoxin, digitoxin, metildigoxin), thrombolytics (e.g. tPA, reteplase, anistreplase, tenecteplase, streptokinase, urokinase), platelet inhibitors (e.g., abciximab, tirofiban, eptifibatide), clopidogrel, ticlopidine, aspirin, dipyridamole, anticoagulants (e.g. heparin, low molecular weight heparin), direct thrombin inhibitors (e.g. hirudin, argatroban, bivalirudin, ximelagatran, melagatran, dabigatran) potassium chloride, clonidine, prazosin, aldose reductase inhibitors (e.g., zopolrestat), antiarrhythmic drugs (e.g. quinidine, procainamide, lidocaine, tocainide, mexiletine, propafenone, amiodarone, clofilium, sotalol, dofetilide, moricizine, flecainide, aprinidine and ajmaline).

Pharmaceutical compositions of the invention may optionally include pharmaceutically acceptable organic or inorganic carrier substances suitable for parenteral, enteral or intranasal application which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

For topical applications, formulations may be prepared in a topical ointment or cream containing one or more compounds of the invention. When formulated as an ointment, one or more compounds of the invention suitably may be employed with either a paraffmic or a water-miscible base. The one or more compounds also may be formulated with an oil-in-water cream base. Other suitable topical formulations include e.g. lozenges and dermal patches.

Intravenous or parenteral administration, e.g., sub-cutaneous, intraperitoneal or intramuscular administration are generally preferred.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. In general, a suitable effective dose of one or more compounds of the invention, particularly when using the more potent compound(s) IMAC inhibitor compounds, will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day. The desired dose is suitably administered once daily, or several sub-doses, e.g. 2 to 4 sub-doses, are administered at appropriate intervals through the day, or other appropriate schedule.

The following non-limiting examples are illustrative of the invention. All documents mentioned herein are incorporated herein by reference.

General Comments

The following method and materials were employed in the examples below.

### METHODS

Experimental Preparation

All procedures involving the handling of animals were approved by the Animal Care and Use Committee of the Johns Hopkins University and adhered with public health service guidelines. As described in detail elsewhere (28), adult guinea pigs (n=48) were anesthetized with pentobarbital sodium (30 mg/kg ip), and retrogradely perfused as Langendorff preparations with oxygenated (95% 02-5% CO₂) Tyrode solution containing (in mmol/l) 130 NaCl, 1.2 MgSO₄, 4.75 KCl, 5.0 dextrose, and 1.25 CaCl₂ (pH 7.40) at 36±1° C. Hearts were stained with the voltage-sensitive dye, di-4-ANEPPS (15 μmol/l) for 10 min, and then positioned in a chamber such that the mapping field was centered over a 0.8 cm diameter region of left ventricular epicardium, midway between apex and base. Gentle pressure was applied to the posterior surface of the heart with a movable piston to stabilize the mapped surface against the imaging window of the chamber allowing us to avoid the use of electromechanical inhibitors, which interfere with repolarization, and the incidence of arrhythmias. Although experiments were typically completed within 1.5 to 2 h, these preparations remained stable for over 4 h of perfusion. Pacing with a silver pin needle placed on the epicardium was performed intermittently and briefly at various times during the experimental protocol in order to obtain a quantitative assessment of epicardial conduction velocity at each time point. Pacing was then turned off in order to determine the intrinsic response of hearts to the ischemia reperfusion protocol independent of exogenous stimulation.

High-resolution Optical Mapping

An optical mapping system was designed with the capability of simultaneously recording transmembrane action potentials (APs) from 464 sites. Emitted fluorescence was collected using a custom designed optical macroscope imaging system consisting of a high numerical aperture photographic lens, a dichroic mirror and an emission filter as previously described in detail (29). Emitted light exiting the detector lens was filtered (>610 nm) and focused onto the photodiode array. Photocurrent from each photodiode underwent current-to-voltage conversion, amplification, band-pass filtering, multiplexing, and digitization (1600 samples/s per channel) with 16-bit precision.

Experimental Protocols

Ischemia-Reperfusion Protocol

Following extraction and staining with di-4-ANEPPS, hearts were stabilized for 30 min. Monitoring of APs, perfusion pressure, coronary flow, and temperature ensured electrical stability in all preparations (n=48) used in this study. Pharmacological agents were delivered to the heart in one of two ways: 1) continuously, during control perfusion and reperfusion, or 2) in a high dose bolus (5-10× the optimal equilibration concentration), injected into the coronaries during the ischemic phase, 5 min before the onset of reperfusion. After stabilization, hearts were either perfused with control tyrode solution (control group) or with tyrode containing the mBzR antagonist 4′-Cl-DZP (32-100 μM), the mBzR agonist FGIN-1-27 (4.6-46 μM), the PTP inhibitor CsA (0.1-1.0 μM), or the sarcolemmal K_{ATP} channel blocker, GLIBEN (10 μM) for 25 min (Procedure 1 described above). Following baseline perfusion at 15-20 mL/min to maintain a perfusion pressure of 60-70 mmHg, flow was completely stopped for 30 min (Ischemia phase), and APs (3.8 s epochs) were recorded at 1-min intervals. Following the ischemia phase, perfusion was restored initially at the same flow rate and then was adjusted slightly to exactly match the perfusion pressure during the baseline perfusion phase (Reperfusion phase). The optimal equilibration concentration of each agent was determined by its effects on ischemia-induced AP shortening, recovery of the AP duration (APD), and/or prevention of arrhythmias upon reperfusion.

In a subset of experiments, 4′-Cl-DZP (320 μM, n=4), FGIN-1-27 (460 μM, n=3), or CsA (1.0 μM, n=3) were delivered to the heart in the form of a high-concentration bolus injection delivered directly into the cannula via a side port (Procedure 2 as described above).

Cardiomyocyte Isolation

Cellular electrophysiological measurements were performed on freshly isolated adult guinea pig ventricular myocytes prepared by enzymatic dispersion, as previously described (17). Imaging and electrophysiological recordings were performed after suspending the cells in a solution containing 140 mM NaCl, 5 mM KCl, 1 mM MgCl₂, 10 mM HEPES, 1 mM CaCl₂, pH 7.4 (adjusted with NaOH), supplemented with 10 mM glucose. The dish containing the cardiomyocytes was equilibrated at 37° C. with unrestricted access to atmospheric oxygen on the stage of a Nikon E600FN upright microscope.

Two-photon Laser Scanning Microscopy

The cationic potentiometric fluorescent dye tetramethylrhodamine methyl ester (TMRM) was used to monitor changes in ΔΨₘ as previously described (8). Images were recorded using a two-photon laser scanning microscope (Bio-Rad MRC-1024MP) with excitation at 740 nm (Tsunami Ti:Sa laser, Spectra-Physics) and the red emission of TMRM was collected at 605±25 nm. Other imaging conditions were as previously described (8). Images were analyzed offline using ImageJ software (Wayne Rasband, National Institutes of Health, http://rsb.info.nih.gov/ij/).

Cardiomyocyte Electrophysiological Studies

Freshly isolated ventricular myocytes, handled as described above, were whole-cell patch-clamped in a flow chamber on the stage of the two-photon microscope using borosilicate glass pipettes (1-4 MΩ tip resistance). APs were recorded in current clamp mode using an Axopatch 200A amplifier coupled to a Digidata 1200A interface (Axon Instruments, Union City, Calif.), as previously described (8). Myocytes were superfused with (in mmol/L) NaCl 140, KCl 5, MgCl₂ 1, CaCl₂ 2, glucose 10, and HEPES 10 (pH 7.4 with NaOH). Intracellular solutions contained (in mmol/L) potassium glutamate 130, KCl 9, NaCl 10, MgCl₂ 0.5, MGATP 5, EGTA 1, and HEPES 10 (pH 7.0 with KOH). APs were evoked by brief (5 ms) current injections applied at 2 s intervals.

Materials

TMRM and di-4-ANEPPS were purchased from Molecular Probes, Inc. 4′-Cl-DZP, FGIN-1-27, CsA, and GLIBEN were obtained from Sigma-Aldrich. Stock solutions of these reagents were prepared in DMSO and concentrated enough to avoid exceeding 0.1% DMSO (vol/vol) in the final solution.

### Example 1

### mBzR Antagonist Stabilizes ΔΨₘ and the Cellular Action Potential

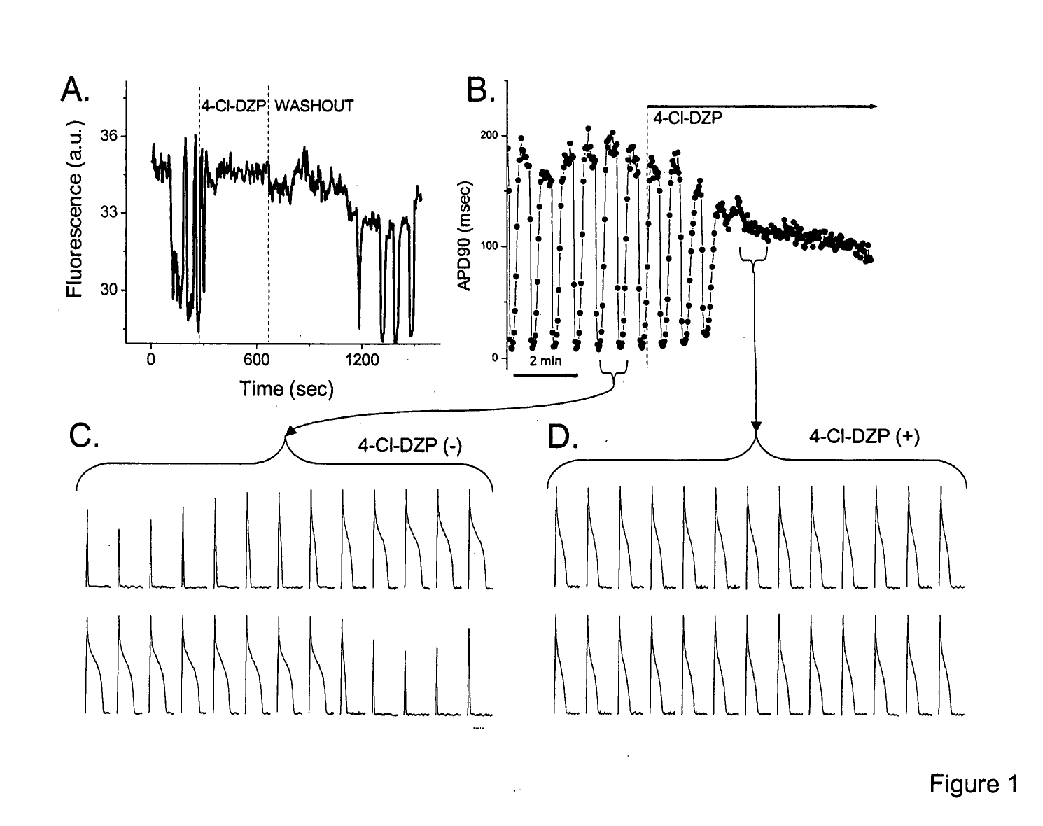

To demonstrate a mechanistic link between the mitochondrial energy state and electrical excitability, the previously described method for triggering whole-cell oscillations in ΔΨₘ by focal two-photon laser excitation was used. In this method, laser-induced depolarization of a few mitochondria leads to a sustained autonomous oscillation in the entire mitochondrial network (8). ΔΨₘ, reported by TMRM fluorescence, was imaged while simultaneously recording Action Potentials (APs) using the patch-clamp technique in current-clamp mode (FIG. 1). The mBzR antagonist 4′-Cl-DZP suppressed whole-cell oscillations in ΔΨₘ within minutes of application and the effect was reversible as ΔΨₘ oscillations returned within 10 min of washout of the compound (FIG. 1A). Through its stabilizing effect on ΔΨₘ, 4′-Cl-DZP also eliminated oscillations in APD (FIG. 1B-D). In the absence of the drug, APD₉₀ decreased to an electrically inexcitable state within ˜4 seconds (2-3 stimuli at 0.5 Hz rate) during each cycle of mitochondrial depolarization. Within ˜2 min of drug application (64 μM; FIGS. 1B and 1D), AP oscillations were suppressed and APD₉₀ stabilized.

“Mitochondrial criticality” refers to the state of the mitochondrial network just prior to cell-wide depolarization of ΔΨₘ, when the system becomes very sensitive to even small perturbations in conditions (20). Since an elevation of ROS and loss of ΔΨₘ also occurs during ischemia and reperfusion of the intact heart, the idea that mitochondrial criticality might contribute to alterations in the electrophysiological substrate leading to post-ischemic arrhythmias in the intact heart was tested.

Baseline Electrophysiological Properties

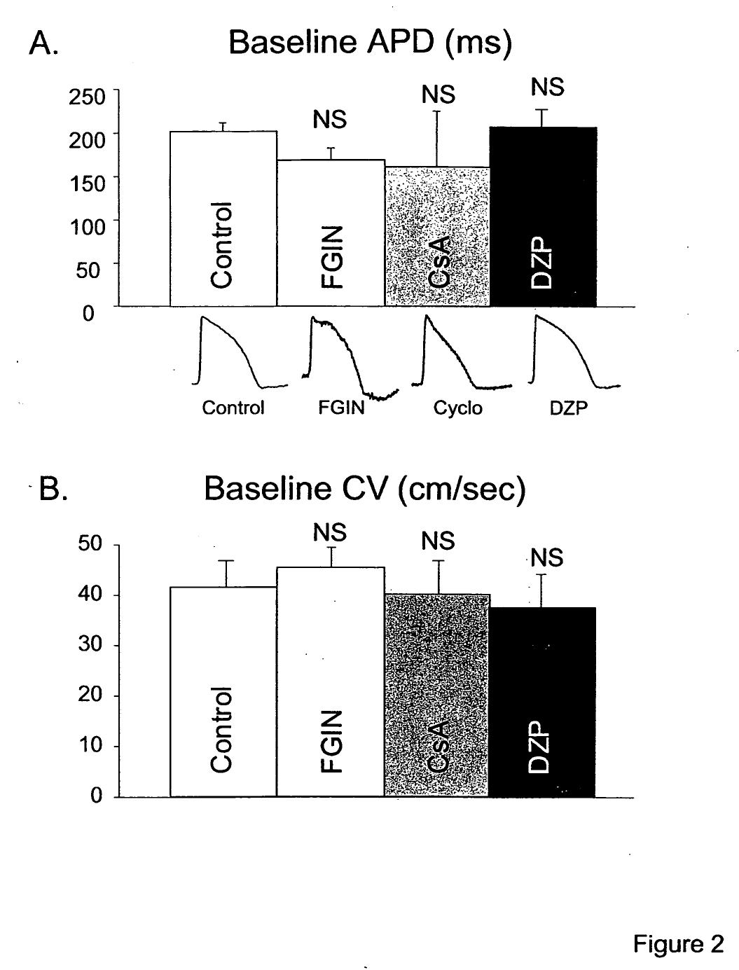

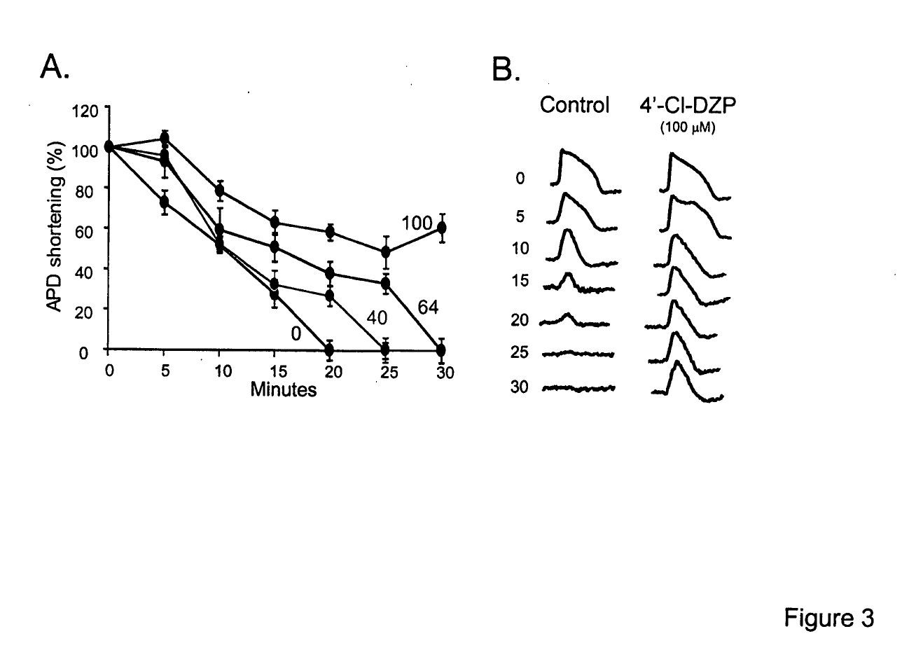

In order to investigate whether mitochondrial ROS-induced activation of IMAC underlies the electrical dysfunction of hearts subjected to IR, a protocol for reproducibly inducing ventricular fibrillation (VF) in Langendorff-perfused guinea pig hearts was established. To rule out any direct effects of the compounds used in the study on sarcolemmal ion channels, it was first determined if 4′-Cl-DZP, CsA, FGIN-1-27, or GLIBEN altered intrinsic electrophysiological properties, including Action Potential Duration (APD) and conduction velocity (CV) during baseline perfusion. The results are shown in FIG. 2. None of the compounds had a significant impact on baseline APD (FIG. 2A) (186±18, 174±16, 168±24, 188±16 ms, 182±16: control, FGIN-1-27, CsA, 4′Cl-DZP, GLIBEN respectively) or CV (FIG. 2B) (41±4, 44±6, 40±4, 39±5, 41±5 cm/sec) at the optimal concentration for each agent used in this study (4.6 μM FGIN-1-27, 0.2 μM CsA, 64 μM 4′Cl-DZP, and 10 μM GLIBEN). At a relatively high concentration (46 μM), the mBzR agonist FGIN-1-27, which was previously shown to induce ΔΨₘ depolarization (8), produced significant APD shortening and membrane inexcitability that occurred within 15 minutes of drug delivery during normal perfusion (results not shown). The highest concentration (100 μM) of 4′Cl-DZP tested had no significant effect on APD or CV during baseline perfusion and was the most effective in blunting AP shortening during ischemia (see FIG. 3 as discussed below); however, it was not as effective as the lower concentration of 64 μM in restoring the APD upon reperfusion and preventing arrhythmias. Thus the lower concentration was selected as the optimal concentration to be used in subsequent experiments.

### Example 2

### Response to Ischemia

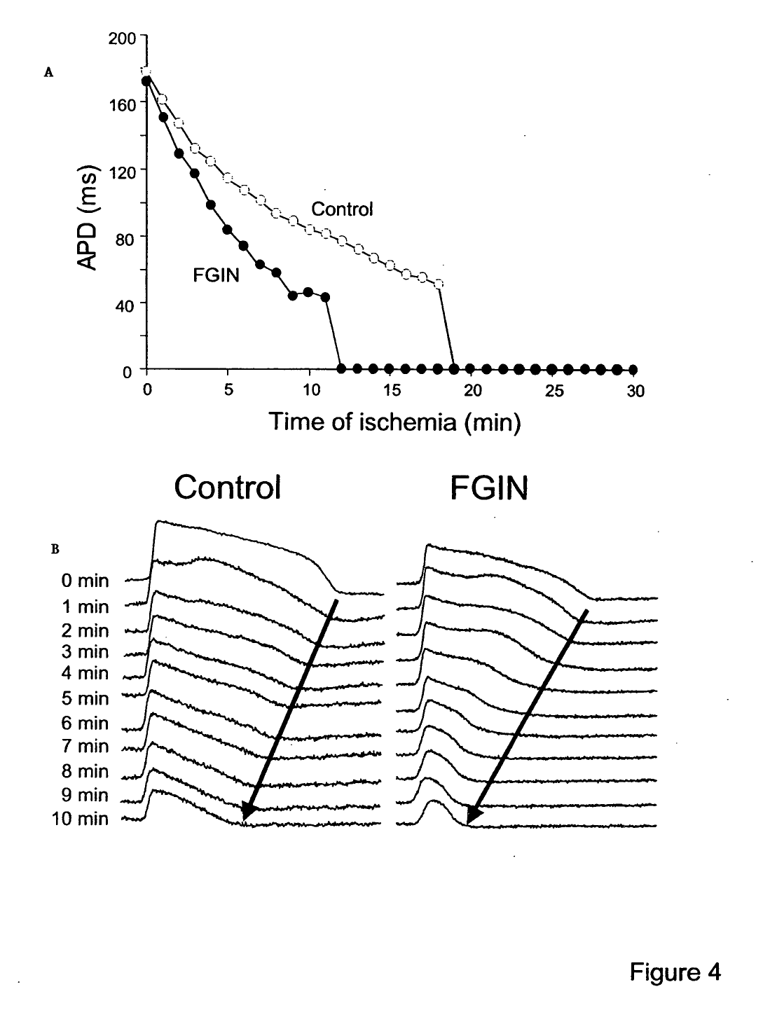

Next, the ability of any of these pharmacological interventions to modulate the electrophysiological response of hearts to no-flow global ischemia was tested. As expected, ischemia resulted in progressive shortening of APD (FIG. 3), resulting in complete loss of the AP after 18.4×3.3 min under control conditions (i.e., without pharmacological intervention prior to the ischemic episode). Also, APD shortening in ischemia was accompanied by progressive reduction of the AP upstroke velocity, which occurred over a similar time frame (see, for example, FIG. 4). Interestingly, treatment with the mBzR antagonist had a profound influence on the electrophysiological response to ischemia. 4′-Cl-DZP blunted APD shortening in a dose-dependent fashion during ischemia, consistent with its proposed role in preventing mitochondrial depolarization and the subsequent activation of sarcolemmal K_{ATP} currents (FIG. 3A). Remarkably, at the highest concentration of 4′-Cl-DZP tested (100 μM), APs persisted even after 30 min of no-flow ischemia, a time when all of the untreated ischemic hearts became electrically inexcitable (FIG. 3B).

Conversely, as shown in FIG. 4, pre-treatment of hearts with the mBzR agonist FGIN-1-27 accelerated APD shortening during ischemia and significantly reduced the time for the onset of inexcitability (12.7±2.6 min) compared to control hearts (˜18 min, FIG. 4A). In addition, FGIN-1-27 treatment was associated with disproportionate slowing of CV compared to untreated hearts. Such profound CV slowing in FGIN-1-27 treated hearts was shortly followed by the emergence of an area of functional conduction block within as little as 10 min of ischemia, while in control hearts, conduction was slowed, but block was not yet established after 10 min of ischemia.

Inhibition of the mitochondrial PTP with CsA did not impact the response of hearts to ischemia, as APD shortening and onset of inexcitability were not significantly altered compared to control hearts (not shown). In accord with the hypothesis that mitochondrial uncoupling was linked to sarcolemmal K_{ATP} channel activation, the K_{ATP} channel inhibitor GLIBEN caused a marked reduction in the rate of APD shortening with ischemia up to a point when the heart abruptly became inexcitable at ˜18 min (not shown). Interestingly, the time to inexcitability was similar to that of control hearts despite profound preservation of APD by GLIBEN at earlier time points (not shown).

Ischemic elevation of extracellular K⁺ concentration, which may be mediated in part by K_{ATP} channel opening, might also be expected to partially depolarize the cellular resting membrane potential. This effect can be indirectly assessed by examining the action potential amplitude (APA) and upstroke velocity (dF/dt), since these parameters are predominantly determined by the extent of inactivation of Na⁺ currents due to depolarization of the resting membrane potential. Therefore, in addition to measuring the marked changes in APD and the time to onset of inexcitability, ischemia-induced changes in the normalized APA were quantified (relative to the pre-ischemia baseline level in each heart), in addition to upstroke velocity (dF/dt) in control hearts and hearts treated with FGIN-1-27, 4′Cl-DZP, GLIBEN, and a combination of FGIN-1-27 and GLIBEN. As expected, ischemia causes progressive reduction of normalized APA and dF/dt. Ischemia-induced reduction of APA and dF/dt was highly modulated by treatment with FGIN-1-27, as shown in FIG. 4, 4′Cl-DZP, and GLIBEN. While FGIN-1-27 accentuated the ischemia-induced reduction of APA and dF/dt, treatment with 4′-Cl-DZP and GLIBEN protected against such decrease in both parameters relative to control hearts. Finally, treatment with GLIBEN was also effective in abolishing the FGIN-1-27-induced reduction of APA and dF/dt when the heart was treated with both compounds.

There was a more pronounced reduction in membrane excitability during ischemia in hearts treated with FGIN-1-27. In a sequence of isopotential contour maps recorded every 1.2 ms, the sequential spread of the AP wavefront across the heart treated with FGIN-1-27 after 11 minutes of ischemia is demonstrated. 11 minutes of ischemia results in conduction block as the depolarization wave front fails to propogate in to the electrically silent area. These areas of electrical silence are also present at the same location in the heart during reperfusion and likely participate in the formation of sustained arrhythmias. A similar pattern of conduction block upon reperfusion is also seen in another heart treated with FGIN-1-27.

### Example 3

### Response to Reperfusion

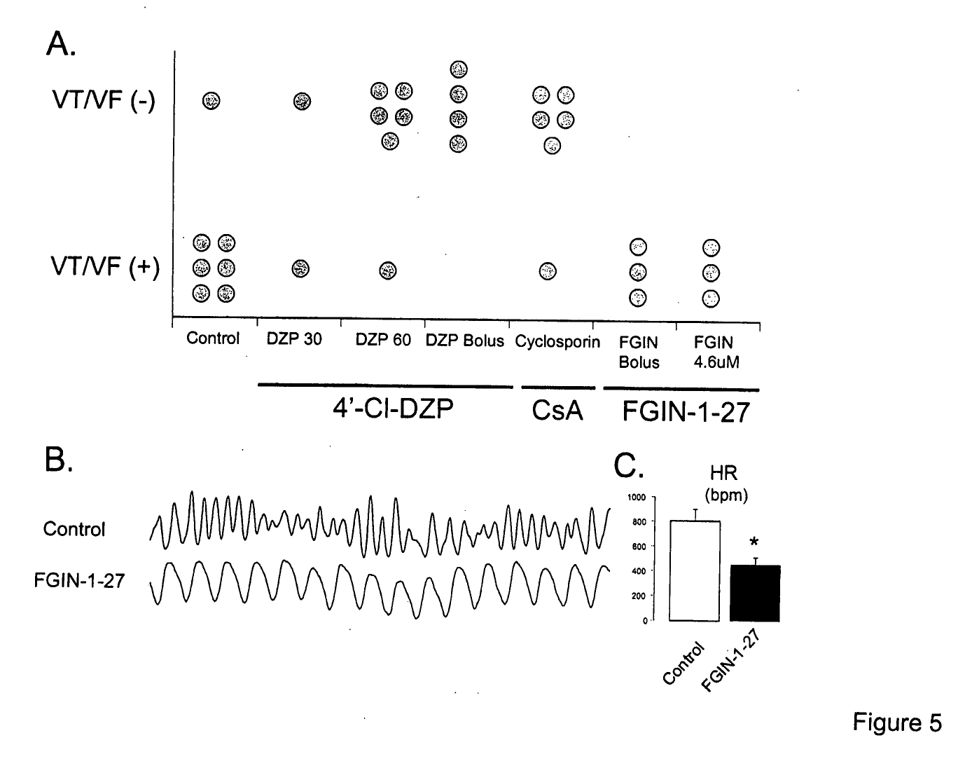

After characterizing the ischemia-induced electrophysiological changes, the response of hearts to reperfusion following the 30 min ischemic episode with and without pretreatment with 4′-Cl-DZP, FGIN-1-27, CsA, GLIBEN, or a combination of FGIN-1-27 and GLIBEN was examined. Reperfusion of untreated control hearts was associated with sustained ventricular fibrillation (VF) in 89% of hearts (FIG. 5). FIG. 5B shows representative AP traces recorded in control and FGIN 1-27 treated hearts.

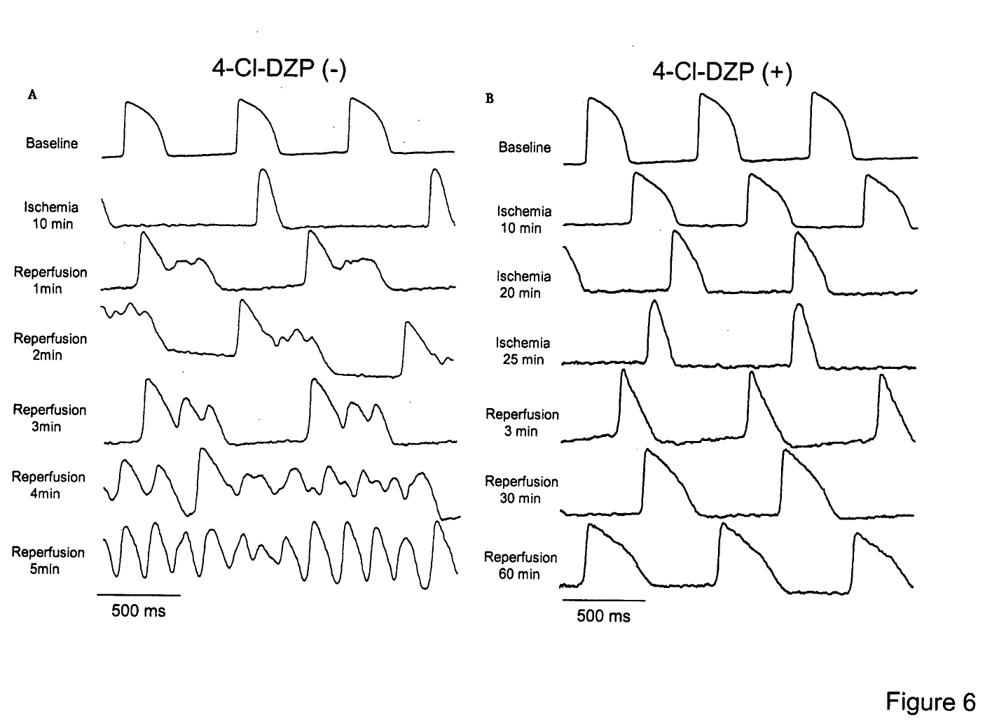

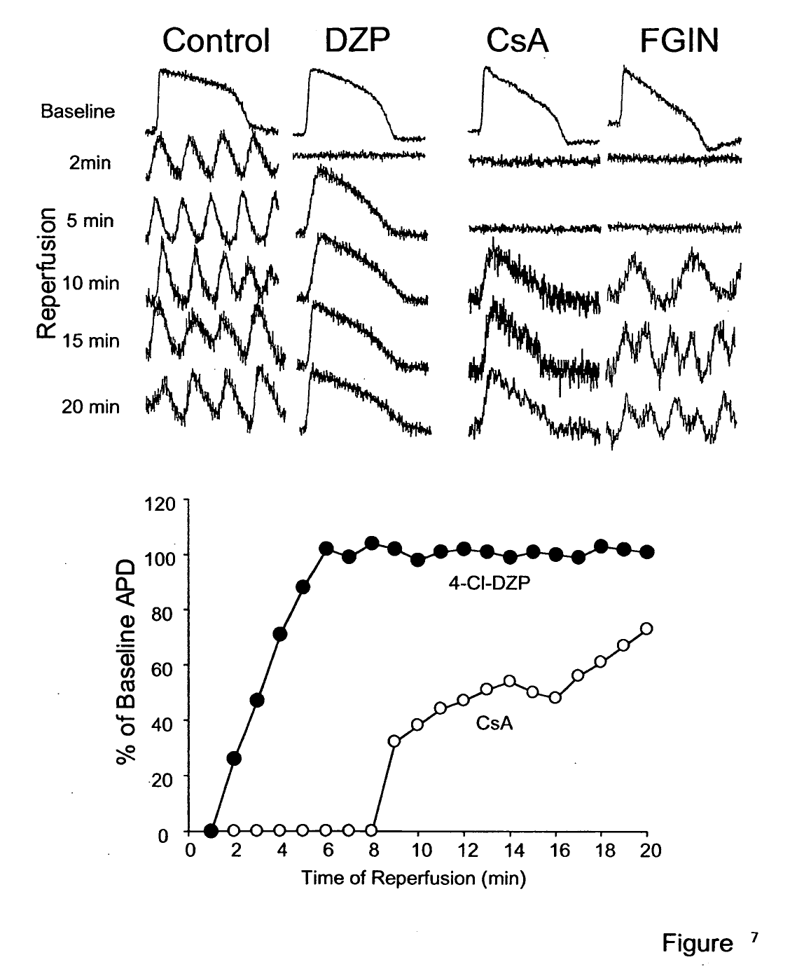

In addition to preventing ischemia-induced APD shortening, treatment of hearts with 4′-Cl-DZP promoted the rapid recovery of the AP morphology and duration upon reperfusion (FIGS. 6 and 7), and markedly decreased the incidence of post-ischemic arrhythmias (10 of 12 hearts exhibited no sustained arrhythmias; FIG. 5). Prevention of reperfusion-related VF was evident both when 4′-Cl-DZP (32-64 μM) was delivered continuously prior to ischemia (6 of 8), or in a high dose (320 μM) bolus (4 of 4) given 5 min prior to the onset of reperfusion (FIG. 5A).

In contrast, FGIN-1-27 treatment resulted in a prolonged period of electrical silence upon reperfusion, followed by ventricular tachycardia (VT) in all (6 of 6) treated hearts (FIGS. 5 and 7). Compared to control hearts, VT presented with a significantly longer cycle length in FGIN-1-27-treated hearts (FIG. 5) compared to polymorphic VT/VF in normal hearts (not shown).

Treatment with 0.2 μM CsA was also associated with a markedly (p<0.01) longer period of electrical inexcitability upon reperfusion (8 min for CsA compared to <2 min for 4′-Cl-DZP), followed by a slower, partial recovery of the AP as compared to that in 4′-Cl-DZP pretreated hearts (FIG. 7). This concentration of CsA was chosen based on an earlier report which demonstrated that it was optimal for inhibiting PTP in the reperfused heart (14). This was confirmed by further experiments using higher (0.4, 1 μM) or lower (0.1 μM) CsA concentrations, which resulted in diminished AP recovery (FIG. 7B) and a higher incidence of VT/VF (FIG. 6A).

Based on these results, a mechanism of conduction block and reentry dependent on the formation of areas of the myocardium undergoing regional or temporal changes in ΔΨₘ, constituting a metabolic current sink, has been identified. In this case, the propagating wave of depolarization encounters clusters of cells in which the mitochondrial network is depolarized and the sarcolemmal K_{ATP} channels are open. These cells are rendered inexcitable because of the large background K⁺ conductance, locking the sarcolemmal membrane potential close to Ek, rather than by their inability to conduct current, i.e., they are powerful current sinks. Consistent with this is the disproportionate reduction in AP amplitude and upstroke velocity recorded during ischemia in FGIN-1-27 treated hearts (FIG. 4). This mechanism would be distinct from existing conduction block models in that the charge of the depolarizing cell would be dissipated into the metabolic sink causing the impulse to decrement and block. In contrast, for conduction block dependent on compromised gap junction function, the charge of the depolarized cell builds up when the block is encountered due to the reduced electrotonic sink and the higher voltage increases the likelihood that the wave of depolarization will bypass the region of block via an alternative conduction path. Thus, gap-junctional block can increase the “safety factor” of conduction (27), whereas metabolic sinkiblock dramatically decreases the safety factor. The latter could lead to a zone of functional block (see, for example, FIG. 5) that extends even beyond the regions containing myocytes with depolarized mitochondria.

In support of the metabolic sink/block hypothesis, the mBzR agonist FGIN-1-27, which promotes ΔΨₘ depolarization (8), not only accelerated APD shortening, but created widespread regions of slowed conduction. It is possible that decreased gap-junctional conductance might also be induced by the collapse of ΔΨₘ, and this will aid in distinguishing between the two possible mechanistic explanations for impaired electrical propagation. Further, APD shortening as well as reduced AP amplitude and upstroke velocity occurring during the first 10 minutes of ischemia were blunted by K_{ATP} channel blockade, as were the effects of FGIN-1-27 on the ischemic response (FIG. 4), indicating that the opening of K_{ATP} channels, secondary to ΔΨₘ depolarization, were responsible for the early electrophysiological remodeling during ischemia.

Electrical excitability beyond 20 minutes of ischemia, however, could only be preserved by blocking the upstream mitochondrial target, thus suggesting that prevention of energy depletion might also prevent changes occurring later in the ischemic period, which could include changes in gap junctional conductance. Inhibition of ΔΨₘ depolarization with 4′-Cl-DZP was also found to be more effective than continuous inhibition of K_{ATP} channels in preventing reperfusion-induced tachyarrhythmias, although a bolus dose of GLIBEN given just prior to reperfusion successfully prevented tachyarrhythmias in 2 of 3 hearts (data not shown). These findings highlight a mechanism involving blocking mitochondrial de-energization above that of inhibiting the downstream effects of metabolism on ion channels.

The results suggest that macroscopic electrical heterogeneity in the post-ischemic heart stems, in part, from instability at the subcellular level. That is, a perturbation of mitochondrial function can lead to failure of the mitochondrial network of the myocyte, regional or temporal alterations in the action potential, zones of impaired conduction and, ultimately, a fatal ventricular arrhythmia.

Further, the above results indicate that mitochondrial criticality plays a key role in the recovery of the electrical activity in the post-ischemic heart. Blocking mitochondrial inner membrane ion channels through mBzR inhibition could prevent mitochondrial ROS-induced ROS release, and the loss of ΔΨₘ triggered by metabolic stress. This effect of the mBzR antagonist was correlated with preservation of the AP during ischemia, as well as restoration of normal electrical activity upon reperfusion.

### CITED DOCUMENTS

The following documents are referred to above by reference to the below sequential numbering with such number designations above generally set forth within brackets (i.e. [ ]).
1. Carmeliet, E. 1999. Cardiac ionic currents and acute ischemia: from channels to arrhythmias. Physiol Rev 79:917-1017.
2. Coronel, R., Wilms-Schopman, F. J., Opthof, T., Cinca, J., Fiolet, J. W., and Janse, M. J. 1992. Reperfusion arrhythmias in isolated perfused pig hearts. Inhomogeneities in extracellular potassium, ST and TQ potentials, and transmembrane action potentials. Circ Res 71:1131-1142.
3. De Groot, J. R., and Coronel, R. 2004. Acute ischemia-induced gap junctional uncoupling and arrhythmogenesis. Cardiovasc Res 62:323-334.
4. Picard, S., Rouet, R., Ducouret, P., Puddu, P. E., Flais, F., Criniti, A., Monti, F., and Gerard, J. L. 1999. KATP channels and ‘border zone’ arrhythmias: role of the repolarization dispersion between normal and ischaemic ventricular regions. Br J Pharmacol 127:1687-1695.
5. Crompton, M., Virji, S., Doyle, V., Johnson, N., and Ward, J. M. 1999. The mitochondrial permeability transition pore. Biochem Soc Symp 66:167-179.
6. Duchen, M. R. 1999. Contributions of mitochondria to animal physiology: from homeostatic sensor to calcium signalling and cell death. J Physiol 516 (Pt 1): 1-17.
7. Romashko, D. N., Marban, E., and O'Rourke, B. 1998. Subcellular metabolic transients and mitochondrial redox waves in heart cells. Proc Natl Acad Sci U S A 95:1618-1623.
8. Aon, M. A., Cortassa, S., Marban, E., and O'Rourke, B. 2003. Synchronized whole cell oscillations in mitochondrial metabolism triggered by a local release of reactive oxygen species in cardiac myocytes. J Biol Chem 278:44735-44744.
9. Ambrosio, G., Zweier, J. L., Duilio, C., Kuppusamy, P., Santoro, G., Elia, P. P., Tritto, I., Cirillo, P., Condorelli, M., Chiariello, M., et al. 1993. Evidence that mitochondrial respiration is a source of potentially toxic oxygen free radicals in intact rabbit hearts subjected to ischemia and reflow. J Biol Chem 268:18532-18541.
10. Griffiths, E. J., Ocampo, C. J., Savage, J. S., Stern, M. D., and Silverman, H. S. 2000. Protective effects of low and high doses of cyclosporin A against reoxygenation injury in isolated rat cardiomyocytes are associated with differential effects on mitochondrial calcium levels. Cell Calcium 27:87-95.
11. Suleiman, M. S., Halestrap, A. P., and Griffiths, E. J. 2001. Mitochondria: a target for myocardial protection. Pharmacol Ther 89:29-46.
12. Kroemer, G., Dallaporta, B., and Resche-Rigon, M. 1998. The mitochondrial death/life regulator in apoptosis and necrosis. Annu Rev Physiol 60:619-642.
13. Griffiths, E. J., and Halestrap, A. P. 1993. Protection by Cyclosporin A of ischemia/reperfusion-induced damage in isolated rat hearts. J Mol Cell Cardiol 25:1461-1469.
14. Halestrap, A. P., Connern, C. P., Griffiths, E. J., and Kerr, P. M. 1997. Cyclosporin A binding to mitochondrial cyclophilin inhibits the permeability transition pore and protects hearts from ischemia/reperfusion injury. Mol Cell Biochem 174:167-172.
15. Weiss, J. N., Korge, P., Honda, H. M., and Ping, P. 2003. Role of the mitochondrial permeability transition in myocardial disease. Circ Res 93:292-301.
16. Berkich, D. A., Salama, G., and LaNoue, K. F. 2003. Mitochondrial membrane potentials in ischemic hearts. Arch Biochem Biophys 420:279-286.
17. O'Rourke, B., Ramza, B. M., and Marban, E. 1994. Oscillations of membrane current and excitability driven by metabolic oscillations in heart cells. Science 265:962-966.
18. O'Rourke, B. 2000. Pathophysiological and protective roles of mitochondrial ion channels. J Physiol 529 Pt 1:23-36.
19. Cortassa, S., Aon, M. A., Winslow, R. L., and O'Rourke, B. 2004. A mitochondrial oscillator dependent on reactive oxygen species. Biophys J 87:2060-2073.
20. Aon, M. A., Cortassa, S., and O'Rourke, B. 2004. Percolation and criticality in a mitochondrial network. Proc Natl Acad Sci U S A 101:4447-4452.
21. DeLorey, T. M., and Olsen, R. W. 1992. Gamma-aminobutyric acidA receptor structure and function. J Biol Chem 267:16747-16750.
22. Garnier, M., Boujrad, N., Oke, B. O., Brown, A. S., Riond, J., Ferrara, P., Shoyab, M., Suarez-Quian, C. A., and Papadopoulos, V. 1993. Diazepam binding inhibitor is a paracrine/autocrine regulator of Leydig cell proliferation and steroidogenesis: action via peripheral-type benzodiazepine receptor and independent mechanisms. Endocrinology 132:444-458.
23. Romeo, E., Auta, J., Kozikowski, A. P., Ma, D., Papadopoulos, V., Puia, G., Costa, E., and Guidotti, A. 1992. 2-Aryl-3-indoleacetamides (FGIN-1): a new class of potent and specific ligands for the mitochondrial DBI receptor (MDR). J Pharmacol Exp Ther 262:971-978.
24. Beavis, A. D. 1992. Properties of the inner membrane anion channel in intact mitochondria. J Bioenerg Biomembr 24:77-90.
25. Beavis, A. D., and Davatol-Hag, H. 1996. The mitochondrial inner membrane anion channel is inhibited by DIDS. J Bioenerg Biomembr 28:207-214.
26. Kinnally, K. W., Zorov, D. B., Antonenko, Y. N., Snyder, S. H., McEnery, M. W., and Tedeschi, H. 1993. Mitochondrial benzodiazepine receptor linked to inner membrane ion channels by nanomolar actions of ligands. Proc Natl Acad Sci U S A 90:1374-1378.
27. Kleber, A. G., and Rudy, Y. 2004. Basic mechanisms of cardiac impulse propagation and associated arrhythmias. Physiol Rev 84:431-488.
28. Akar, F. G., Roth, B. J., and Rosenbaum, D. S. 2001. Optical measurement of cell-to-cell coupling in intact heart using subthreshold electrical stimulation. Am J Physiol Heart Circ Physiol 281 :H533-542.
29. Akar, F. G., Spragg, D. D., Tunin, R. S., Kass, D. A., and Tomaselli, G. F. 2004. Mechanisms underlying conduction slowing and arrhythmogenesis in nonischemic dilated cardiomyopathy. Circ Res 95:717-725.

The invention has been described in detail with particular reference to the preferred embodiments thereof. However, it will be appreciated that modifications and improvements within the spirit and teachings of the inventions may be made by those in the art upon considering the present disclosure.

1. A method for treating ischemia-related arrhythmias, comprising administering one or more IMAC inhibitor compounds to a mammal suffering from or susceptible to an ischemia-related arrhythmia.

2. The method of claim 1 wherein the mammal has been identified as suffering from or susceptible to ischemia-related arrhythmia.

3. The method of claim 1 further comprising identifying the one or more compounds as IMAC inhibitor compounds.

4. The method of claim 1 wherein the mammal is undergoing a surgical procedure.

5. The method of claim 1 wherein the mammal has a predisposition to being susceptible to an ischemia-related arrhythmia.

6. The method of claim 1 wherein the mammal has been identified as suffering from or susceptible to chronic ischemia.

7. The method of claim 1 wherein the one or more IMAC inhibitor compounds prevent mitochondrial depolarization by at least about 30 percent in a mitochondrial fluorescence assay.

8. The method of claim 1 wherein one or more IMAC inhibitor compounds reduces or eliminates the events of ischemia comprising AP shortening and/or membrane inexcitability.

9. The method of claim 1 wherein one or more IMAC inhibitor compounds reduces or eliminates the events of reperfusion comprising ventricular fibrillation.

10. The method of claim 1 wherein the one or more compounds comprise 4′-chlorodiazepam, PK11195, amiodarone, amitryptyline, imipramine, dibucaine, propranolol, quinine, clonazepam, bupivacaine, etidocaine, pindolol, or timolol.

11. The method of claim 1 wherein the mammal is a human.

12. A method for treating ischemia-related arrhythmias, comprising:
a) identifying a subject as suffering from or susceptible to ischemia-related arrhythmia;
b) administering to the identified subject one or more IMAC inhibitor compounds.

13. The method of claim 12 further comprising identifying the one or more compounds as IMAC inhibitor compounds.

14. The method of claim 12 wherein the one or more IMAC inhibitor compounds prevent mitochondrial depolarization by at least about 30 percent in a mitochondrial fluorescence assay.

15. The method of claim 12 wherein the one or more compounds comprise 4′-chlorodiazepam, PK11195, amiodarone, amitryptyline, imipramine, dibucaine, propranolol, quinine, clonazepam, bupivacaine, etidocaine, pindolol, or timolol.

16. A method for modulating membrane potential of cardiac cells, comprising:
administering one or more IMAC inhibitor compounds to cardiac cells, wherein membrane potential is modulated.

17. The method of claim 16 wherein the cardiac cells have irregular electrical properties at about the time of administration.

18. The method of claim 16 wherein the cardiac cells are susceptible to irregular electrical properties.

19. The method of claim 16 wherein the one or more IMAC inhibitor compounds prevent mitochondrial depolarization by at least about 30 percent in a mitochondrial fluorescence assay.

20. A method for modulating membrane potential of cardiac cells, comprising administering one or more IMAC agonist compounds to cardiac cells, wherein membrane potential is modulated.

21. The method of claim 20 wherein the one or more IMAC agonist compounds promotes mitochondrial depolarization by at least about 30 percent in a mitochondrial fluorescence assay.

22. The method of claims 20 wherein the one or more compounds comprise 4′-chlorodiazepam, PK11195, amiodarone, amitryptyline, imipramine, dibucaine, propranolol, quinine, clonazepam, bupivacaine, etidocaine, pindolol, or timolol.
